(19) United States
(12) Patent Application Publication
(10) Pub. No.: US 2008/0234477 A1
(43) Pub. Date: Sep. 25, 2008

(54) Method for preparing chitosan nano-particles

(75) Inventors: Yau Shan Szeto, Kowloon (CN); Zhigang Hu, Kowloon (CN)

Correspondence Address: The Hong Kong Polytechnic University;Suite 600, 1800 Diagonal Road, Mailbox # 119, Alexandria, VA, 22314, US

(73) Assignee: The Hong Kong Polytechnic University, Hong Kong (HK)

(21) Appl. No.: 12/153,833

(22) Filed: May 27, 2008

(30) Foreign Application Priority Data
Sep. 26, 2006 (CN) 2000610139605.4
Sep. 24, 2007 (IB) PCT/IB2007/002922

(51) Int. Cl. C07H 1/00; C07H 1/06

(52) U.S. Cl. 536/123.1; 536/127

(57) ABSTRACT

This invention involves a new method for preparing chitosan nano-particles. This method uses the nano-cavity technology so as to obtain pure chitosan nano-particles and pure chitosan nano-particle latex in a simple way.

This application hereby claims priority to PRC Application 200610139605.4.

### TECHNICAL FIELD

This invention involves a new method for preparing chitosan nano-particles. This method uses the nano-cavity technology so as to obtain pure chitosan nano-particles and pure chitosan nano-particle latex in a simple way.

### BACKGROUND

Currently, most of the methods for preparing chitosan nano-particles are based on chemically crosslinked deposition or oil/water inverse crosslinkage, and chemical dispersers are used in some of these methods. Some methods, though very few, obtain pure chitosan particles through mechanical grinding, e.g. China Patent Application CN200410089449.6. Besides, these, there are few methods that use ultrasonic cavity technology for preparing chitosan nano-particles, e.g. China Patent Application CN200410021761.1; however, this application still uses chemical aids such as ion cross-linking agent and surfactant.

It is an object of the present invention to overcome the disadvantages and problems in the prior art.

### Details of Invention

The purpose of this invention is to provide a new method for preparing chitosan nano-particles. This method uses the nano-cavity technology so as to obtain pure chitosan nano-particles and pure chitosan nano-particle dispersion latex containing only water and chitosan in a simple way.

To fulfill the above purpose, the method for preparing chitosan nano-particle latex of this invention includes the following steps:

(1) Dissolve chitosan in a diluted acid solution to obtain a chitosan solution;

(2) Allow the chitosan in the solution to deposit rapidly with a kind of alkaline solution;

(3) Wash the deposit with deionized water until the filtrate exhibits neutral property;

(4) Subject the chitosan-containing deionized water solution to ultrasonic cavity treatment to crush the depositing in it into nano-particles, so that pure chitosan nano-particle latex is obtained.

The method for preparing chitosan nano-particles of this invention also includes the step of filtrating or drying the chitosan nano-particle latex obtained with the above method in order to obtain the chitosan nano-particles.

This invention also provides a method for preparing chitosan nano-particle latex that coexists with other nano-particles. This method follows Step (1) of the method for preparing chitosan nano-particle latex and further includes the step of dispersing the other nano-particles in the chitosan solution obtained in Step (1).

This invention also provides a method for preparing chitosan nano-particles that coexist with other nano-particles. This method includes the step of filtrating or drying the chitosan nano-particle latex that coexists with other nano-particles prepared above.

This invention further provides a method for preparing chitosan nano-particle latex that coexists with water-soluble substances. This method follows Step (1) of the method for preparing chitosan nano-particle latex and further includes the step of dissolving water-soluble substances in the chitosan solution obtained in Step (1) in order to obtain a chitosan nano-particle latex with slow-release effect.

This invention also provides a method for preparing chitosan nano-particles that coexist with water-soluble substances. This method includes the step of filtrating or drying the chitosan nano-particle latex that coexists with water-soluble substances prepared above.

This invention also provides a method for preparing chitosan nano-particle latex that coexists with transient metal oxides. This method follows Step (1) of the method for preparing chitosan nano-particle latex and further includes the steps as follows: Dissolve the inorganic salt of the transition metal in the chitosan solution prepared in Step 1. Agitate mechanically until the chitosan molecules chelate with the transition metal ions. And then proceed with the following steps. The transition metal oxides described above include but are not limited to silver oxide.

Soluble transition metal inorganic salts such as silver nitrate, platinum chloride and HAuCl₄ can be used in this method. With the reducing power of the chitosan to the transition metal ions in the transition metal inorganic salts, nano-scale transition metal oxide particles are generated directly during the agitation, and finally composite nano-particles combining chitosan nano-particles and transition metal oxide nano-particles are obtained. This invention also provides a method for preparing chitosan nano-particles that coexist with transition metal oxides. This method includes the step of filtrating or drying the chitosan nano-particle latex that coexists with transition metal oxides prepared above.

This invention also provides the nano-particles prepared with any of the above methods.

### Specific Implementation Mode

The following is to, in conjunction with the specific implementation modes and implementation examples, describe the method for preparing chitosan nano-particle latex of this invention in detail. The examples described are only for illustrative description and should not be interpreted that this invention is limited to this.

The method for preparing chitosan nano-particle latex described for this invention can include:

(1) Dissolve 0.5-5 g of chitosan solid in 100-500 ml of 0.3-1% acetic acid solution under mechanical agitation;

(2) Adjust the pH value of the above chitosan solution to more than 11 (5 ml/min) with strong ammonia liquor (25 g/100 ml) under rapid mechanical agitation (e.g. 300 rpm or more) to obtain white chitosan deposit. Age it for 0.5-1 hour under the ambient temperature;

(3) Filtrate the chitosan deposit obtained under reduced pressure. Elute the white chitosan deposit repeatedly with deionized water until the filtrate exhibits neutral property;

(4) Transfer the dried chitosan deposit into a conical beaker and add deionized water to total volume of 100-500 ml. Place the conical beaker in an ice-water bath. Place the probe of an ultrasonic cell crusher in the conical beaker, with the probe tip at 0.5-2 cm below the surface of the liquid. Select proper output power and ultrasonic time depending on the size of the nano-particles needed and perform ultrasonic crushing, and then pure chitosan nano-particle latex is obtained.

The size of the nano-particles is mainly determined by the actual supersonic power (output power and actual treatment volume) and the intrinsic properties of the chitosan (e.g. molecular weight, crystallinity, etc.). In a certain range, the crystallinity and particle size of the chitosan decreases with the actual power. The particle size distribution mainly depends on the ultrasonic time. The longer the ultrasonic time, the more concentrated the distribution (in a certain range).

In order to obtain chitosan nano-particles, the chitosan nano-particle latex obtained above can be filtrated and/or dried with an already-known appropriate method. The appropriate methods include but are not limited to lyophilization, ultracentrifugal, spray drying, etc.

### EXAMPLE 1

Dissolve 0.3 g of chitosan (degree of deacetylation: 95%; molecular weight: 100000) solid in 100 ml of 0.3% acetic acid solution under mechanical agitation;

Adjust the pH value of the above chitosan solution to more than 11 (5 ml/min) with strong ammonia liquor (25%) (or sodium hydroxide or potassium hydroxide, 3-5 g/100 ml water) under rapid mechanical agitation (500 rpm or more) to obtain white chitosan deposit. Age it for 2 hour under the ambient temperature;

Filtrate the chitosan deposit obtained under reduced pressure. Elute the white chitosan deposit repeatedly with deionized water until the filtrate exhibits neutral property;

Then transfer the dried chitosan deposit into a 100 ml conical beaker and add deionized water to total volume of 100 ml. Place it in an ice-water bath. Place the 6 mm probe of an ultrasonic cell crusher, such as Sonics VCX 130 (Sonics & Materials, Inc.) to 0.5 cm below the surface of the liquid. With the output power of 25 W, select different ultrasonic time to obtain nano-particles of different sizes. Refer to Table 1 for the relationship between the ultrasonic time and the particle size (viscosity average molecular weight of chitosan: 1.2×10⁶ Da; degree of deacetylation: 63%).

TABLE 1
Relationship between ultrasonic time and particle
size

| | Ultrasonic time | Particle size |
|---|---|---|
| Sample | (min.) | (nanometer) |
| 1 | 5 mins. | 609 ± 5 |
| 2 | 10 mins. | 507 ± 6 |
| 3 | 15 mins. | 458 ± 5 |
| 4 | 20 mins. | 413 ± 8 |
| 5 | 30 mins. | 386 ± 4 |
| 6 | 40 mins. | 334 ± 5 |

To invest the relationship between the ultrasonic output power and the particle size, during the supersonic cavity treatment, fix the ultrasonic time at 20 mins and select different ultrasonic output power to obtain particles of different sizes. Refer to Table 2 for the relationship between the output power and the nano-particle size (viscosity average molecular weight of chitosan: 1.0×10⁶ Da; degree of deacetylation: 56%).

TABLE 2
Relationship between output power and particle
size

| | | Particle size |
|---|---|---|
| Sample | Output power | (nanometer) |
| 7 | 15 W | 368 ± 5 |
| 8 | 20 W | 362 ± 7 |
| 9 | 25 W | 326 ± 7 |

### EXAMPLE 2

Dissolve 0.3 g of chitosan (degree of deacetylation: 95%; molecular weight: 100000) solid in 100 ml of 0.3% acetic acid solution under mechanical agitation;

Under mechanical agitation (300 rpm), add 5 ml of 5% silver nitrate aqueous solution quickly into the above chitosan solution. Continue agitating for 4 hours until the solution becomes slightly red, and then add 5% sodium hydroxide (or potassium hydroxide) solution quickly until deposit is obtained.

Filtrate the chitosan deposit obtained under reduced pressure. Elute the brown chitosan deposit repeatedly with deionized water until the filtrate exhibits neutral property;

Then transfer the dried chitosan deposit into a 100 ml conical beaker and add deionized water to total volume of 100 ml. Place it in an ice-water bath. Place the 6 mm probe of an ultrasonic cell crusher, such as Sonics VCX 130 (Sonics & Materials, Inc.) to 0.5 cm below the surface of the liquid. With the output power of 25 W, perform ultrasonic treatment for 20 minutes to obtain the chitosan nano-particle latex that exists with nano silver oxide, in which the particle size of the mixture of silver oxide particles and chitosan particles is approximately 300 nm.

This implementation example illustrates the method for preparing chitosan nano-particle latex that coexists with transition metal oxides. This method mainly makes use of the reducing power of the chitosan to the transition metal ions to reduce the transition metal ions into nanocrystal of certain size in the chitosan solution, and then, through alkaline treatment, oxides the transition metal nanocrystal generated to stop its growth while allowing chitosan to deposit. Finally, latex is obtained through ultrasonic treatment.

### EXAMPLE 3

Dissolve 0.3 g of chitosan (degree of deacetylation: 95%; molecular weight: 100000) solid in 100 ml of 0.3% acetic acid solution under mechanical agitation;

Under mechanical agitation (300 rpm), add 0.05 g of titanium dioxide nano-particles (with size of approx. 20 nm) into the above chitosan solution. Continue agitating for 4 hours until the titanium dioxide nano-particles are dispersed sufficiently in the chitosan solution. Then add sodium hydroxide or potassium hydroxide solution (5 g/100 ml water) quickly to obtain white deposit.

Filtrate the chitosan deposit obtained under reduced pressure. Elute the white chitosan deposit repeatedly with deionized water until the filtrate exhibits neutral property;

Then transfer the dried chitosan deposit into a 100 ml conical beaker and add deionized water to total volume of 100 ml. Place it in an ice-water bath. Place the 6 mm probe of an ultrasonic cell crusher, such as Sonics VCX 130 (Sonics & Materials, Inc.) to 0.5 cm below the surface of the liquid. With the output power of 25 W, perform ultrasonic treatment for 20 minutes to obtain the chitosan nano-particle latex that exists with titanium dioxide nano-particles, in which the particle size of the mixture of titanium dioxide nano-particles and chitosan particles is approximately 300 nm.

This implementation example illustrates the method for preparing chitosan nano-particle latex that coexists with other nano-particles. This method disperses other existing nano-particles (solid powder or acidic water latex) into the chitosan solution, allowing the chitosan to settle down and envelop the existing nano-particles, then the latex can be obtained through washing and ultrasonic treatment.

### EXAMPLE 4

Dissolve 0.3 g of chitosan (degree of deacetylation: 95%; molecular weight: 100000) solid in 100 ml of 0.3% acetic acid solution under mechanical agitation;

Under mechanical agitation (300 rpm), add 0.05 g of Vitamin C powder into the above chitosan solution. Continue agitating until the Vitamin C dissolves completely in the chitosan solution. Then add 5% sodium hydroxide (or potassium hydroxide) solution quickly to obtain white deposit.

Filtrate the chitosan deposit obtained under reduced pressure. Elute the white chitosan deposit repeatedly with deionized water until the filtrate exhibits neutral property;

Then transfer the dried chitosan deposit into a 100 ml conical beaker and add deionized water to total volume of 100 ml. Place it in an ice-water bath. Place the 6 mm probe of an ultrasonic cell crusher, such as Sonics VCX 130 (Sonics & Materials, Inc.) to 0.5 cm below the surface of the liquid. With the output power of 25 W, perform ultrasonic treatment for 20 minutes to obtain the chitosan nano-particle latex, in which the particle size of the chitosan particles containing Vitamin C is approximately 300 nm.

A method for preparing chitosan nano-particle latex that coexists with a water soluble substance is to disperse and dissolve the water soluble substance into the chitosan solution and then to allow the chitosan to settle down. Finally, latex is obtained through washing and ultrasonic treatment.

1. A method for preparing chitosan nano-particles latex, comprising the steps:
dissolving chitosan in a diluted acid solution to obtain a chitosan solution;
allowing said chitosan in the chitosan solution to deposit rapidly with a kind of alkaline solution;
washing said deposit with deionized water until it exhibits neutral property;
subjecting said chitosan-containing deionized water solution to ultrasonic cavity treatment to crush the deposit in it into nano-particles; and
obtaining a pure chitosan nano-particle latex.

2. The method in claim 1, wherein said diluted acid used when dissolving chitosan is acetic acid solution, allowing the chitosan to deposit rapidly involves adjusting the pH of said chitosan solution to more than 11 with an alkaline solution under rapid agitation of 300 rpm to obtain a white chitosan deposit, wherein said alkaline solution can be ammonia liquor, sodium hydroxide solution or potassium hydroxide solution, and subjecting said chitosan-containing deionized water solution to ultrasonic cavity treatment further comprises placing said chitosan-containing deionized water in an ice-water bath, with a probe tip of an ultrasonic cell crusher placed at 0.5-2 cm below the surface of the deionized water solution.

3. The method in claim 1, further comprising the step of filtrating the chitosan nano-particle latex.

4. The method of claim 1, further comprising the step of dissolving said water-soluble substances in the chitosan following dissolving said chitosan.

5. The method of claim 1, further comprising by the step of filtrating the obtained chitosan nano-particle latex that coexists with water soluble substances.

6. The method of claim 1, further comprising the step of dissolving an inorganic salt of a transition metal in said chitosan solution, and agitating mechanically until chitosan molecules chelate with the transition metal ions.

7. The method of claim 6, further comprising the step of filtrating the chitosan nano-particle latex obtained that coexists with transition metal oxides.

8. The method of claim 1, further comprising the step of disposing other nano-particles in said chitosan solution.

9. The method of claim 8, further comprising the step of filtrating said chitosan nano-particle latex that coexists with other nanoparticles.

10. Pure chitosan latex nanoparticle made in accordance with claim 1.
